(19) United States
(12) Patent Application Publication
(10) Pub. No.: US 2006/0275269 A1
(43) Pub. Date: Dec. 7, 2006

(54) Method and data system for the treatment at birth of a patient who is identified at birth as having an elevated risk of a previously unidentified disorder

(76) Inventor: Thomas E. Moore, Tucson, AZ (US)

Correspondence Address: PILLSBURY WINTHROP SHAW PITTMAN LLP, P.O. BOX 10500, MCLEAN, VA, 22102, US

(21) Appl. No.: 11/146,941

(22) Filed: Jun. 6, 2005

(51) Int. Cl. A61K 35/14

(52) U.S. Cl. 424/93.7

(57) ABSTRACT

The present invention provides a procedure used in the overall treatment of a patient who is identified at birth as having an elevated risk of a previously unidentified disorder and method of using a data system to assist in the procedure. In a preferred embodiment, the procedure involves, at the birth of the patient, making a first time determination that the patient is at an elevated risk of a previously unidentified disorder based on a metric of some type. If it is determined by a medical professional, based upon the metric, that the patient has an elevated risk for a previously unidentified disorder that could benefit from treatment using the patient's own cord blood stem cells, the cord blood stem cells of the patient are collected at birth. After this collection, the collected cord blood stem cells of the patient are available for the subsequent use on the patient at an appropriate time.

### BACKGROUND OF THE INVENTION

1. Field of the Invention

The present invention relates to a method and data system for the treatment of a patient who is identified at birth having an elevated risk of a previously unidentified disorder.

2. Description of the Related Art

Many studies exist on the beneficial aspects of human cord blood stem cells. Many such studies infuse cord blood stem cells into other animals. For instance, as described by Chen J, Sanberg P, Li Y, Wang L, Lu M, Willing A, Sanchez-Ramos J, Chopp M. “Intravenous Administration Of Human Umbilical Cord Blood Reduces Behavioral Deficits After Stroke In Rats” 2001; 32: 2682-2688, the authors tested whether intravenously infused human cord blood stem cells entered the brain, survived, differentiated, and improved neurological functional recovery after a stroke in rats, and showed that human cord blood stem cells did have advantages.

Much more limited studies have been done using humans and human cord blood stem cells. With respect to treatments using human cord blood stem cells in humans, whether there are advantages to treating a patient with their own cord blood stem cells for the purpose of improving their neurological disorder has recently been studied, and preliminary indications are that such treatment may be beneficial.

Studies in which cord blood stem cells are used to treat other disorders are also being performed. Research studies of this type, however, are not directed to standardizing birthing treatment procedures or whether saving cord blood stem cells in a particular case could be beneficial for the infant from whom the cord blood stem cells were collected.

Thus, while such studies suggest there are apparent advantages to treatments using human cord blood stem cells, heretofore there has not been any standardized procedure to ensure that any patient at birth can benefit from the usage of their own cord blood stem cells if, at birth, that patient is identified as having an elevated risk of a disorder that could benefit from usage of cord blood stem cells.

While cord blood stem cell banks usable to store cord blood are well known, these banks have been used in a manner in which, prior to birth, a parent decides that cord blood stem cells will be collected, and as a result of that decision prior to birth, arrangements are prepared in advance to ensure that the cord blood stem cells can be collected at birth, for possible use later in time. A background on such banks, and one particular type of mixed used bank, is described in U.S. Pat. No. 5,993,387 entitled “Computer-Based Mixed-Use Registry Of Placental And Umbilical Cord Stem Cells,” which is assigned to the same assignee as the present invention. In the '387 patent, there is described a computer-based mixed-use cord stem cells registry system. The cord stem cells, or a fraction thereof, are stored in a bank for the potential use of the donor and potentially the actual family of the donor child or from an unrelated person for whom the cord stem cells are a match.

Common practice currently, however, is that unless the parents of the newborn have made prior arrangements for the storage of the cord blood stem cells of their newborn, the cord blood stem cells are thrown away.

The present invention, therefore, sets forth a procedure to ensure that any patient at birth can benefit from the usage of their own cord blood stem cells to treat a patient that presents at birth as having an elevated risk of a disorder, as well as a method and data system to assist in such procedure.

### SUMMARY OF THE INVENTION

The present invention provides a procedure used in the overall treatment of a patient who is identified at birth as having an elevated risk of a previously unidentified disorder and method of using a data system to assist in the procedure

In a preferred embodiment, the procedure involves, at the birth of the patient, making a first time determination that the patient is at an elevated risk of a previously unidentified disorder based on a metric of some type. If it is determined by a medical professional, based upon the metric, that the patient has an elevated risk for a previously unidentified disorder that could benefit from treatment using the patient's own cord blood stem cells, the cord blood stem cells of the patient are collected at birth. After this collection, the collected cord blood stem cells of the patient are available for the subsequent use on the patient at an appropriate time.

In a preferred embodiment, the method of using the data system to assist in the procedure involves providing availability of a cord blood stem cell kit, such as extra inventory, at a birthing facility. At birth a determination is made by a medical professional, based upon a metric, whether the patient has an elevated risk for a previously unidentified disorder that could benefit from treatment using the patient's own cord blood stem cells. A series of steps are then implemented to ensure that the cord blood stem cell kit is used to collect cord blood stem cells of the patient, and that identification for the patient and the collected cord blood stem cells is obtained, preferably by entry into a computer system at the birthing facility. Thereafter, the collected cord blood stem cells are transferred from the birthing facility to a processing facility, where the collected cord blood stem cells are processed or manipulated, and/or stored for use at a subsequent appropriate point in time.

Other embodiments, aspects, and advantages of the present invention are described further herein.

### BRIEF DESCRIPTION OF THE DRAWINGS

These and other aspects and features of the present invention will become apparent to those of ordinary skill in the art upon review of the following description of specific embodiments of the invention in conjunction with the accompanying figures, wherein:

FIG. 1 illustrates a flow chart of the procedure used in the overall treatment of a patient who is identified at birth as having an elevated risk of a previously unidentified disorder according to the present invention; and

FIGS. 2A and 2B illustrate a flow chart of a method of using the data system to assist in the procedure according to the present invention.

### DETAILED DESCRIPTION OF THE PREFERRED EMBODIMENTS

The inventions disclosed herein describe a procedure used in the overall treatment of a patient who is identified at birth as having an elevated risk of a previously unidentified disorder, and a method of using a data system to assist in the procedure.

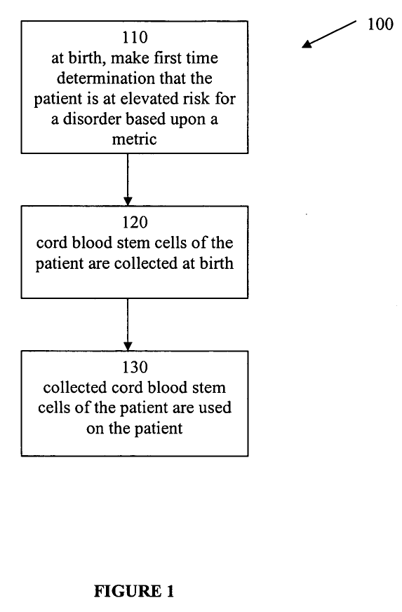

FIG. 1 illustrates a procedure 100 according to the present invention. According to the procedure, as shown, in step 110, at the birth of the patient, a first time determination is made by a medical professional, such as a doctor, that the patient has an elevated risk for a previously unidentified disorder of some type that could benefit from the usage of the patient's own cord blood stem cells, based upon a metric. Type of disorders can be neurological, such as a cerebral disorder, ischemic encephalopathy, cerebral palsy, or other abnormal neurological condition, respiratory, or of other types, including damaging events that occur at birth. Since all newborns are at some risk of having disorders, by elevated risk is meant a higher than normal perceived risk of a disorder of some type that is potentially treatable using cord blood stem cells.

If it is determined that the patient has an elevated risk for a previously unidentified disorder, step 120 follows and cord blood stem cells of the patient are collected at birth. After an appropriate period of time that can vary widely, in step 130, the collected cord blood stem cells of the patient are used on the patient. Certain details of these steps will now be described in further detail.

It is first noted, however, that the procedure 100 of the present invention requires planning in advance, since the cord blood stem cells are viable for a period of only about 10-20 minutes after birth. Accordingly, unless the determination step 110 and the collection step 120 occur within this viable period, the stem cells are lost, and will be of no medical use thereafter.

The step 110 determinations by a medical professional can use different metrics, including a combination of different metrics. And the metric used will preferably err on the side of caution, since other than cost there is no downside to storing samples of the cord blood stem cells. Examples of different metrics that can be used by a medical professional to determine that collection should take place include an APGAR score, a subset of an APGAR score, such as only activity, appearance and respiration, a cord blood ph less than 7.0, negative analysis of blood gases (cord blood or other blood gases), or evidence of metabolic acidemia on the cord ph by base deficit. Still even simpler metrics, such as an FLK (“funny looking kid”) metric can be used.

With regard to using an APGAR score as a metric, this can be further specified in terms of the APGAR score changes over time, as is known. For example, a determination can be made that the cord blood stem cells should be collected based on an APGAR score of 3 or less at an interval of between about 1 and 5 minutes, preferably either 1 or 5 minute intervals.

With respect to the collection step 120, in-vivo collection is normally used, typically within the first 5 minutes of birth. Ex-vivo collection is also possible, typically in situations in which the mother's life is also in jeopardy, a caesarian birth is required, or other complications have presented.

With respect to step 130, when this step occurs can vary. In certain circumstances, the use will take place very soon after birth, such as within one week, given that birth is the time that the determination of their being a possible disorder, and, for instance, an early infusion of the cord blood stem cells into the same patient soon after the initial determination can be beneficial. In other circumstances, the use will not take place for some time thereafter, typically after symptoms of the disorder present themselves.

With respect to this method, it is noted that certain disorders can be very expensive to treat later in life, and the cost of caring for patients with certain presently non-treatable neurological disorders can be very high. As such, given these high costs, the method described herein has the potential for significant cost savings if uniformly implemented. Furthermore, since the usage of the cord blood stem cells is strictly autologous, the issues, including significant safety issues, which surround non-autologous do not present themselves. The collection for potential subsequent autologous usage is unique relative to the other general types of collections, related or donation or research.

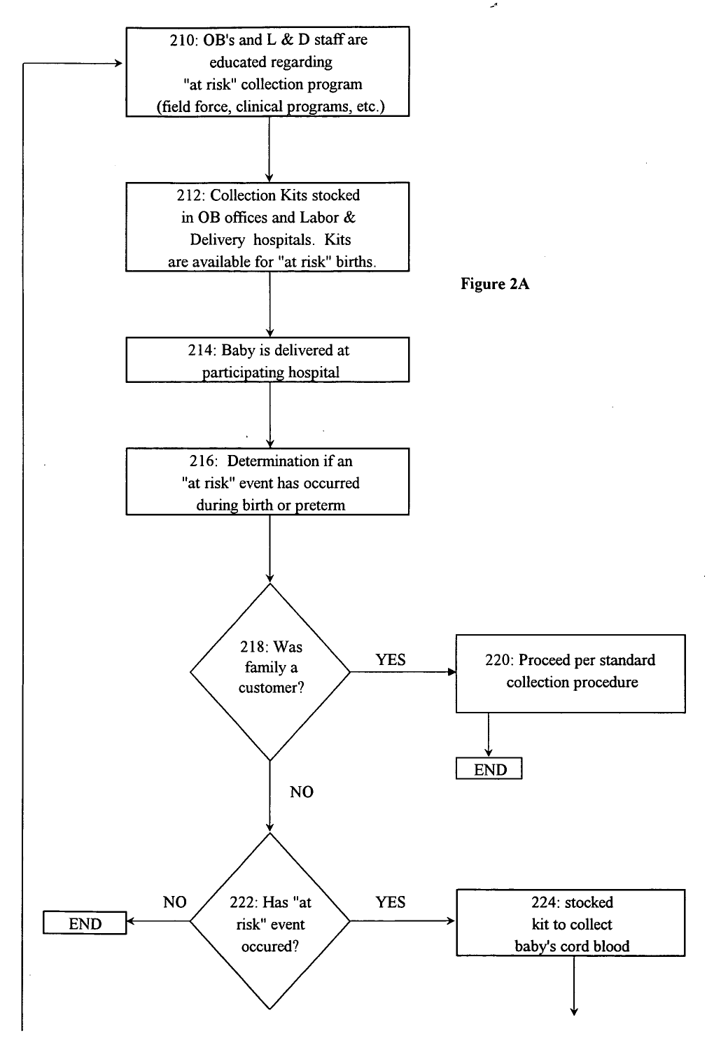

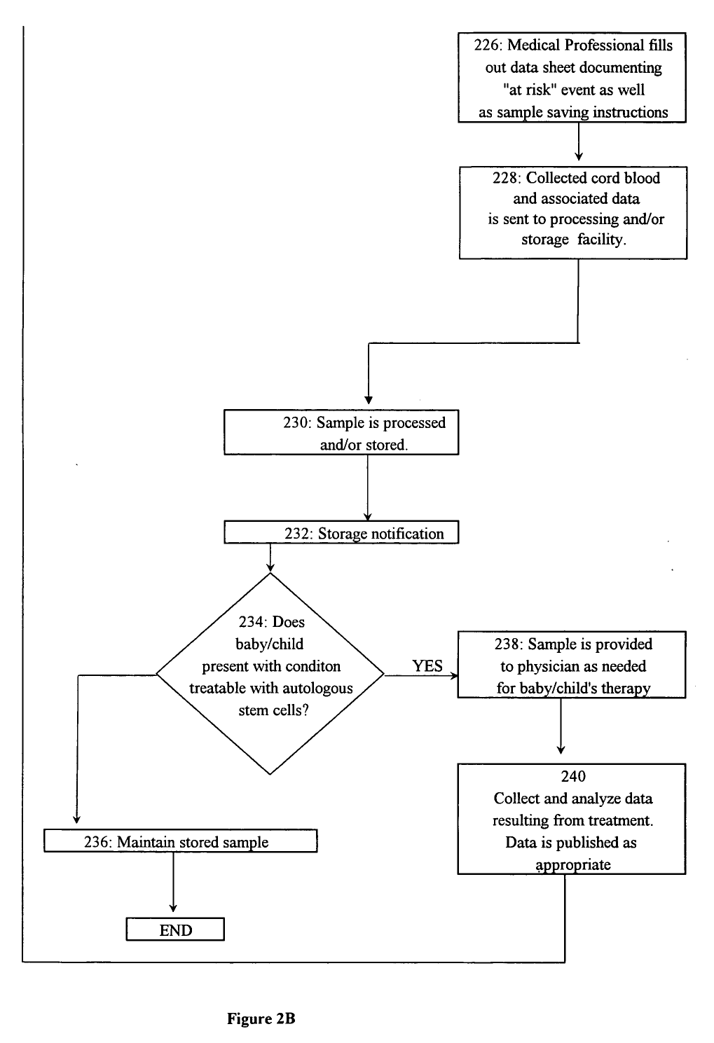

FIGS. 2A and 2B illustrates a flow chart of a preferred embodiment of a method 200 of the procedure according to the present invention. In a preferred embodiment, the method 200, in an initial step 210, Medical professionals, such as obgyn's, labor and delivery (L&D) staff are educated in the “at-risk” nature of certain birth that are treatable using cord-blood stem cells, as well as how to properly collect cord blood stem cells using a stem cell collection kit. Such education is needed since at present, professionals who work at birthing facilities, whether private clinics or hospitals, are not aware of the advantages that stem cells have for certain disorders, and particularly disorders that present themselves for the first time at the birth of a newborn or show a likelihood of developing in the future based on a metric.

Thereafter, in step 212, extra collection kits are stocked in the inventory of an obgyn's, hospital labor and delivery units, and other birthing facilities, or nearby location where one can be timely delivered, so that there is availability. Stem-cell collection kits that are preferably used can be obtained from Cord Blood Registry, Inc., at www.cordblood.com. The computer system described hereinafter can preferably be used to track inventory at different birthing facilities.

Step 214 illustrates that a baby is delivered at a hospital or other birthing facility, with step 216 following, and the medical professional making a determination of whether there is an “at-risk” birth, based upon the metrics described above. Step 218 follows, in which a determination is made whether there was already a plan to store the cord blood stem cells for this newborn. If there was, then step 220 follows and the procedures that were previously already in place for such collection are implemented.

Much more typically, however, there was not a plan to store the cord blood stem cells, and step 222 will follow, in which a decision is made based upon the step 218 determination: has an “at-risk” event occurred? If not, then the process ends, and the cord blood stem cells are discarded per normal procedures. If so, however, then step 224 follows, and one of the stocked cord blood stem cell kits is used to collect the baby's cord blood, preferably upon receiving parental consent.

Step 226 follows, and the medical professional or staff fills out a data sheet documenting an “at-risk” event as well as sample saving instructions. This data collection can be implemented in a number of ways, such as using paper forms that are then sent along with the cord blood, as described hereinafter. A preferred method is to keep track of the cord blood stem cells on a computer system. The computer system used can be a modification of the computer system described in U.S. Pat. No. 5,993,387 entitled “Computer-Based Mixed-Use Registry Of Placental And Umbilical Cord Stem Cells,” and as such this patent is expressly incorporated by reference herein. Modifications made include adding fields to the database to account for the various test results and determinations made as described herein, which given the teaching herein, will be apparent to one of ordinary skill.

Step 228 follow, in which the collected cord blood stem cells are then sent to storage, which can be a processing facility for longer term storage, or a unit of a hospital for an infusion a short time period after birth.

Step 230 follows, and the collected cord blood stem cells are sent to a processing facility for processing or manipulating and/or storage. By processing is meant procedures such as concentrating the stem cells, in which the original identity of the stem cells is maintained. In contrast, by manipulating is meant procedures in which the original identity of the stem cells is changed. Once any procedures are finished and the collected cord blood stem cells are stored, a notification is preferably provided to the medical professional and the family of this storage, shown by step 232.

Step 234, shown as coming after step 232 since the decision in step 234 will typically be made after the storage occurs, is the determination if the baby/child presents with a condition that is treatable with autologous stem cells. If not, then step 236 follows and stored cord blood stem cells will remain stored. If so, however, then in step 238 the stored cord blood stem cells are provided to the physician for use as needed, in many instances at a time that corresponds to when symptoms present. At this time, parental consent is also obtained to use the cord blood stem cells for the autologous use. The cord blood stem cells are then typically used by infusion of the stored cord blood stem cells into the patient from whom they were obtained. Step 240 follows and a determination is made how and if to analyze and publish data associated with the decision to use the collected cord blood stem cells, which can be provided, for instance, at subsequent education meetings previously described in step 210.

Although the present invention has been particularly described with reference to embodiments thereof, it should be readily apparent to those of ordinary skill in the art that various changes, modifications and substitutes are intended within the form and details thereof, without departing from the spirit and scope of the invention. Accordingly, it will be appreciated that in numerous instances some features of the invention will be employed without a corresponding use of other features. Further, those skilled in the art will understand that variations can be made in the number and arrangement of components illustrated in the above figures. It is intended that the scope of the appended claims include such changes and modifications.

What is claimed is:

1. A method to assist in medically treating a human patient comprising:
at birth of the patient, making a first time determination based upon a metric that the patient is at an elevated risk for a previously unidentified disorder; and
collecting cord blood stem cells of the patient at birth if it is determined that the patient has the elevated risk for the disorder based upon the metric.

2. The method according to claim 1 further including the step of storing the collected cord blood stem cells.

3. The method according to claim 2 further including the steps of
determining that the autologous use of the collected cord blood stem cells will be beneficial for the patient; and
using the collected cord blood stem cells of the patient for the patient some time after the step of determining.

4. The method according to claim 3 wherein the steps of determining and using take place within about two months of the patient's birth.

5. The method according to claim 1 further including the steps of:
processing the collected cord blood stem cells to obtain processed cord blood stem cells; and
storing the processed cord blood stem cells.

6. The method according to claim 5 further including the steps of
determining that the autologous use of the processed cord blood stem cells will be beneficial for the patient; and
using the processed cord blood stem cells of the patient for the patient some time after the step of determining.

7. The method according to claim 5 wherein the step of processing includes concentrating the collected cord blood stem cells.

8. The method according to claim 1 further including the steps of:
manipulating the collected cord blood stem cells to obtain manipulated cord blood stem cells; and
storing the manipulated cord blood stem cells.

9. The method according to claim 8 further including the steps of
determining that the autologous use of the manipulated cord blood stem cells will be beneficial for the patient; and
using the manipulated cord blood stem cells of the patient for the patient some time after the step of determining.

10. The method according to claim 1 wherein the previously unidentified disorder is a neurological disorder.

11. The method according to claim 1 wherein the previously unidentified disorder is a respiratory disorder.

12. The method according to claim 1 wherein the metric is a subset of an APGAR score.

13. The method according to claim 12 wherein the subset of the APGAR score includes a combination of APGAR variable components.

14. The method according to claim 13 wherein activity, appearance and respiration components of the APGAR score are used as the metric.

15. The method according to claim 1 wherein the metric is an APGAR score.

16. The method according to claim 15 wherein the step of collecting determines that the cord blood stem cells should be collected based on the metric of an APGAR score of 3 or less at an interval of between about 1 and 5 minutes.

17. The method according to claim 1 wherein the metric is cord blood ph less than 7.0.

18. The method according to claim 1 wherein the metric is evidence of metabolic acidemia on the cord ph by base deficit.

19. The method according to claim 1 wherein the metric is a negative analysis of blood gases.

20. A method to assist in medically treating each a human patient comprising the steps of:
providing availability of a cord blood stem cell kit for use at a birthing facility;
making a first time determination whether the patient is at an elevated risk for a previously unidentified disorder based upon a metric upon birth of the patient at the birthing facility;
using the cord blood stem cell kit to collect cord blood stem cells of the patient; and
obtaining information on the patient, the determination, and an identification of the collected cord blood stem cells.

21. The method according to claim 20 wherein the steps of making, using and obtaining are performed on a plurality of different patients, each using a different one of a plurality of cord blood stem cell kits that are available from the step of providing.

22. The method according to claim 20 further including the steps of:
transferring the collected cord blood stem cells from the birthing facility to a processing facility; and
storing the collected cord blood stem cells at the processing facility as stored cord blood stem cells.

23. The method according to claim 22 further comprising the steps of
obtaining permission to use the collected stem cells on the patient;
updating the information to note the permission received;
transferring the stored cord blood stem cells to a treatment facility; and
at the treatment facility, using the stored cord blood stem cells of the patient for the patient and updating the information to reflect such using.

24. The method according to claim 22 further including the step of processing or manipulating the collected cord blood stem cells prior to the step of storing.

25. The method according to claim 22 wherein a computer system is used to during the steps of obtaining and updating.

26. The method according to claim 20 wherein the metric is a subset of an APGAR score.

27. The method according to claim 26 wherein the subset of the APGAR score includes a combination of APGAR variable components.

28. The method according to claim 27 wherein activity, appearance and respiration components of the APGAR score are used as the metric.

29. The method according to claim 20 wherein the metric is an APGAR score.

30. The method according to claim 29 wherein the step of collecting determines that the cord blood stem cells should be collected based on the metric of an APGAR score of 3 or less at an interval of between about 1 and 5 minutes.

31. The method according to claim 20 wherein the metric is cord blood ph less than 7.0.

32. The method according to claim 20 wherein the metric is evidence of metabolic acidemia on the cord ph by base deficit.

33. The method according to claim 1 wherein the metric is a negative analysis of blood gases.
